(19) United States
(12) Patent Application Publication
(10) Pub. No.: US 2006/0233701 A1
(43) Pub. Date: Oct. 19, 2006

(54) Method and apparatus to improve the industrial production of hydrogen-carbon monoxide

(76) Inventors: Thomas Parias, Chicago, IL (US); Raja Amirthalingam, Downers Grove, IL (US); Omar Germouni, Chicago, IL (US)

Correspondence Address: Linda Russel;Air Liquide, Intellectual Property Dept., 2700 Post Oak Blvd., Suite 1800, Houston, TX, 77056, US

(21) Appl. No.: 11/389,518

(22) Filed: Mar. 24, 2006

Related U.S. Application Data
(60) Provisional application No. 60/666,349, filed on Mar. 30, 2005.

(51) Int. Cl. C01B 3/02; C07C 27/26

(52) U.S. Cl. 423/648.1; 518/726

(57) ABSTRACT

Embodiments of the invention provide a method, article of manufacture and apparatus for controlling production of hydrogen. In one embodiment, the method includes measuring an amount of hydrogen present at a first point in a hydrogen production process using a palladium-based hydrogen sensor, measuring one or more first production process variables for the hydrogen production process using an additional sensor, inputting the amount of hydrogen and the one or more additional production process variables into a process controller, and modifying one or more second production process variables for the hydrogen production process using a process control system.

### CROSS-REFERENCE TO RELATED APPLICATIONS

This application claims the benefit under 35 U.S.C. §119(e) to provisional application No. 60/666,349, filed Mar. 30, 2005, the entire contents of which are incorporated herein by reference.

### BACKGROUND

Many industries such as the steel, oil refining, chemicals, glass, electronics, healthcare, food processing, metallurgy, paper and aerospace industries utilize industrial gases such as oxygen, nitrogen, hydrogen, and synthetic gas (syngas). Typically, such industrial gases are produced from a plant which may utilize natural resources (e.g., natural gas) as a base material for producing the gases. For example, one type of plant, a Hydrogen/Carbon Monoxide (HyCO) plant may produce purified hydrogen, carbon monoxide, and/or a mixture of both called syngas.

Producing syngas at a plant is a highly complex process that requires extensive control efforts to achieve desired yields of the gases produced while minimizing operating costs of the plant. In order to better control production of the gases, sensors may be utilized to determine the presence and quantity of certain gases at certain points in the production process.

For example, gas chromatographs are typically used in HyCO plants to measure various organic components such as carbon monoxide (CO), carbon dioxide (CO₂), and methane (CH₄). However, when using gas chromatography (GC) the frequency with which the gases can be measured is very low due to the complexity of the GC process and reliability of the measurement is often questionable due to drift and calibration issues. Where the sampling rate is low and the reliability of the measurement is questionable, the type of control that can be applied may be limited (e.g., some types of control may require more sensitive or more frequent measurements to be performed effectively) and thus the achievable control of the overall production process may be decreased. Furthermore, such gas chromatographs may involve high capital cost and the maintenance cost of these traditional analyzers is also high due to the skilled workforce and specific procedures and material it requires. Thus, in some cases, the number of gas chromatograph sensors that can be used in a typical plant may be limited.

So far the replacement of gas chromatographs by other sensor types (e.g., electrochemical or semiconductor sensors) has been limited due to the poor performance of the other sensor types. Some of the main issues are the life time and the stability as well as the cross sensitivity to other gases. For example, where hydrogen is measured in the presence of carbon monoxide, the carbon monoxide may interfere with the accuracy of the measurement (e.g., the measurement may indicate a combined response to the amount of carbon monoxide and the amount of hydrogen without allowing a separate measurement of the hydrogen amount if it is not partly masked).

Accordingly, what is needed is an improved method and apparatus for sensing a gas and controlling gas production processes.

### SUMMARY

Embodiments of the invention provide a method, apparatus, and computer-readable medium for controlling production of hydrogen. In one embodiment, the method includes measuring an amount of hydrogen present at a first point in a hydrogen production process using a palladium-based hydrogen sensor, measuring one or more first production process variables for the hydrogen production process using an additional sensor, inputting the amount of hydrogen and the one or more additional production process variables into a process controller, and modifying one or more second production process variables for the hydrogen production process using a process control system.

### SUMMARY

Embodiments of the invention also provide a computer-readable medium including a program which, when executed by a processor, performs a method for controlling production of hydrogen. The method includes measuring an amount of hydrogen present at a first point in a hydrogen production process using a palladium-based hydrogen sensor, measuring one or more first production process variables for the hydrogen production process using an additional sensor, inputting the amount of hydrogen and the one or more additional production process variables into a process controller, and modifying one or more second production process variables for the hydrogen production process using a process control system.

Embodiments of the invention also provide a system including a palladium-based hydrogen sensor and a multi-variable predictive controller configured to measure an amount of hydrogen present at a first point in a hydrogen production process using a palladium-based hydrogen sensor, measure one or more first production process variables for the hydrogen production process using an additional sensor, input the amount of hydrogen and the one or more additional production process variables into a process controller, and modify one or more second production process variables for the hydrogen production process using a process control system.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a block diagram depicting a hydrogen/carbon monoxide (HyCO) plant according to one embodiment of the invention;

FIG. 2 is a flow diagram depicting a process for utilizing sensor according to one embodiment of the invention; and

FIG. 3 is a graph depicting a predicted value of a process variable according to one embodiment of the invention;

FIG. 4 is a block diagram depicting the control of process variables in a steam methane reforming unit according to one embodiment of the invention; and

FIG. 5 is a block diagram depicting control of a membrane separation unit according to one embodiment of the invention.

### DESCRIPTION OF PREFERRED EMBODIMENTS

Embodiments of the invention provide a method, article of manufacture and apparatus for controlling production of hydrogen. In one embodiment, the method includes measuring an amount of hydrogen present at a first point in a hydrogen production process using a palladium-based hydrogen sensor, measuring one or more first production process variables for the hydrogen production process using an additional sensor, inputting the amount of hydrogen and the one or more additional production process variables into a process controller, and modifying one or more second production process variables for the hydrogen production process using a process control system. By controlling the production process using a palladium-based hydrogen sensor, fuel consumption in the process may be reduced, yields of the process may be increased, and energy consumptions of the process may be reduced.

### EXAMPLES

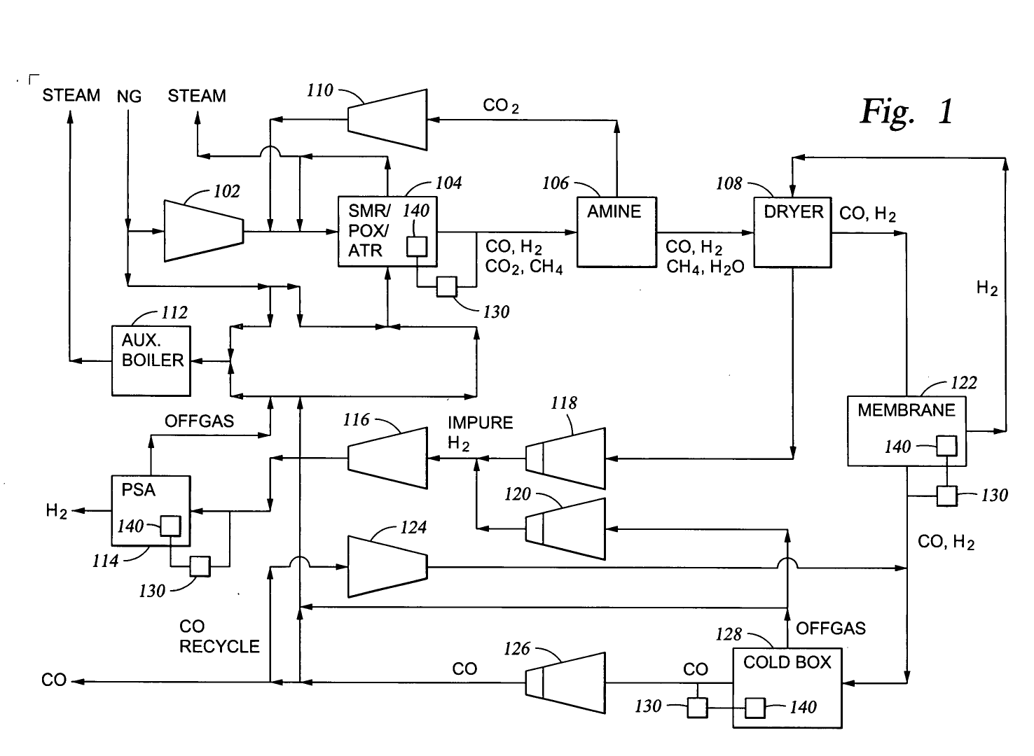

FIG. 1 is a block diagram depicting a hydrogen/carbon monoxide (HyCO) plant 100 according to one embodiment of the invention. As depicted, the plant 100 may receive natural gas (NG) which is compressed by compressor 102. The natural gas may then be processed in a steam methane reformer (SMR), partial oxidation (POX), or auto-thermal reforming (ATR) unit 104 (referred to hereinafter as the SMR unit 104). The SMR may use the natural gas in addition to steam (H₂O) provided by the auxiliary boiler 112 and/or other sources to produce a gas mixture of carbon monoxide, hydrogen, and carbon dioxide (CH₄).

The gas mixture produced by the SMR unit 104 may then be processed by an amine scrubber 106 to remove carbon dioxide from the gas mixture. A portion of the carbon dioxide may be compressed by compressor 110 and fed back to the SMR unit 104. A gas mixture including carbon monoxide, hydrogen, methane, and steam may be produced at the output of the amine scrubber 106 and fed into a dryer 108. The dryer 108 may remove the steam from the gaseous mixture to produce a first gas mixture of hydrogen and methane and a second gas mixture of carbon monoxide and hydrogen. The first gas mixture produced by the dryer 108 may be passed through compressor 118. The second gas mixture produced by the dryer 108 may be passed through a membrane separation unit 122. The membrane separation unit 122 may further reduce the hydrogen content of the second gas mixture and return the separated hydrogen to the dryer 108 for use in drying the gas mixture produced by the amine scrubber 106. The membrane separation unit 122 may also provide a gas mixture of carbon monoxide and hydrogen to a cold box which separates the carbon monoxide from hydrogen and any other gases in the gas mixture produced by the membrane separation unit 122.

The cold box may produce a purified stream of carbon monoxide to compressor 126, the output of which may be output by the plant 100, e.g., to customers which utilize the carbon monoxide. Offgas produced by the cold box 128 (including, e.g., hydrogen and other gaseous impurities) may be compressed by compressor 120, the output of which may be combined with impure hydrogen output by compressor 118. The impure hydrogen output by compressors 118, 120 may be further compressed by compressor 116, the output of which is processed by a pressure swing adsorption (PSA) unit 114. The PSA unit 114 may produce purified hydrogen which may be output by the plant 100 to customers which utilize the purified hydrogen. The PSA unit 114 may also produce offgas which is used by the auxiliary boiler 112 to produce steam for the SMR unit 104.

As described below, the HyCO plant 100 may utilize one or more sensors 130 to measure hydrogen present at various points of the production process. One more of the measurements provided by the sensors 130 may then be input into a controller 140 which utilizes the measurements in addition to one or more other measurements to control an aspect of the production process.

### The Sensor

In one embodiment of the invention, each sensor 130 may be a palladium-based internal hydrogen gas sensor. For example, the sensor 130 may use a palladium-based alloy such as palladium-nickel alloy or a palladium-gold alloy to detect the presence of hydrogen where the palladium is the sensitive component of the alloy and the other component(s) may be used to increase the palladium stability. In some cases, such a hydrogen sensor may have a low cross-sensitivity to the presence of other gases (e.g., the presence of other gases such as carbon monoxide may not interfere with the accuracy of the hydrogen measurement) and may also have a sampling time (e.g., time between samples) of one to three seconds. Such sensors may also measure hydrogen levels over wide concentration ranges with levels ranging from a few parts per million (ppm) to 100%. The sensor 130 may also be inexpensive, require less frequent calibration, require less maintenance, and be provided in a small integration package which would fit the particular physical constraint of the plant design. In some cases, the sensor 130 may be a sensor available from H2Scan, LLC or Makel Engineering, Inc.

In one embodiment of the invention, the sensor 130 may contain a heating element which may be used to adjust the temperature of the sensor 130. The temperature of the sensor 130 may be adjusted, for example, to change the sensitivity of the sensor 130 to the presence of hydrogen. Also, the temperature of the sensor 130 may be adjusted, for example, to decrease the sensitivity of the sensor with respect to carbon monoxide in the gas mixture being measured. Where the temperature of the sensor 130 is adjusted to decrease the sensitivity of the sensor 130 with respect to carbon monoxide in the gas mixture being measured, the temperature of the sensor 130 may be adjusted and maintained above a threshold temperature value. In some cases, the threshold temperature may be determined during a calibration process after the sensor 130 has been installed.

In one embodiment of the invention, the sensor 130 may include an additional layer deposited over the palladium alloy used to sense the concentration of hydrogen. The additional layer may, for example, use size exclusion of molecules to prevent the sensor 130 from inadvertently measuring the presence of other gases such as carbon monoxide. Thus, the additional layer may act as a filter to remove gases which the filter may be cross-sensitive to, such as carbon monoxide.

### Use of the Sensor

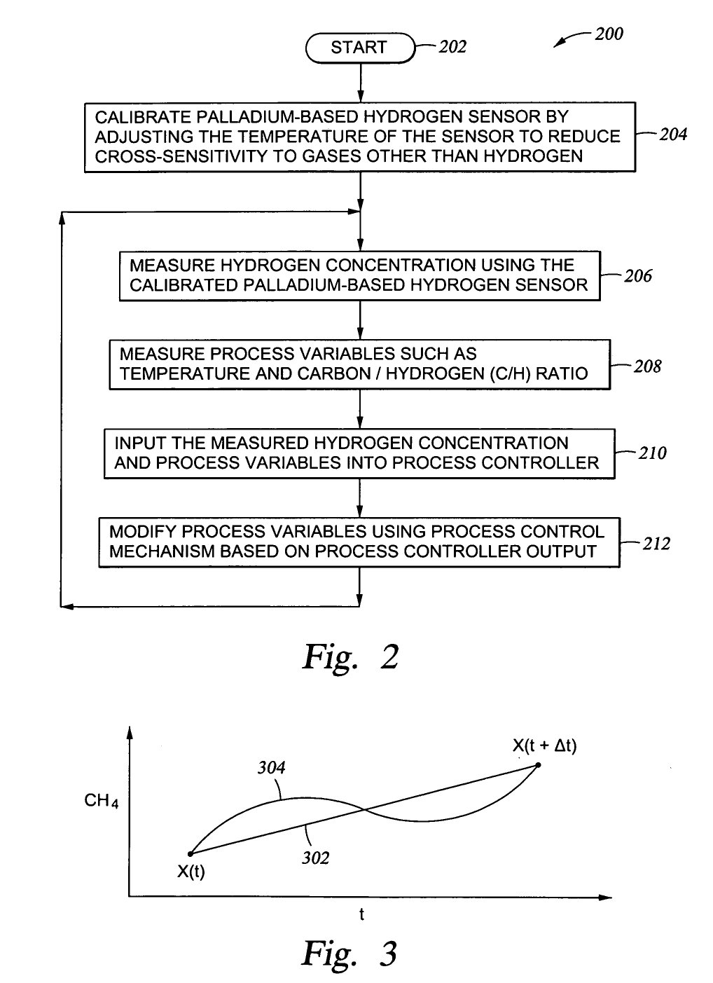

FIG. 2 is a flow diagram depicting a process 200 for utilizing a palladium-based hydrogen sensor 130 in a HyCO plant 100 according to one embodiment of the invention. As depicted, the process 200 may begin at step 202 and continue to step 204 where the palladium-based hydrogen sensor 130 is calibrated by adjusting the temperature of the sensor 130 to reduce cross-sensitivity of the sensor 130 to gases other than hydrogen in the mixture of gases being measured by the sensor 130.

At step 206, the sensor 130 may be used to measure the hydrogen concentration at a point in the HyCO plant 100 (e.g., at a step of the production process). At step 208, process variables such as temperatures and carbon/hydrogen (C/H) ratios for the production process may also be measured. At step 210, the measured hydrogen concentration and the measured process variables may be input into a process controller 140. Then, at step 212, process variables may be modified based on the output of the process controller 140 using process control mechanisms or systems. For example, as described below, the process variable may be a temperature of tubes of catalyst in the SMR unit 104 and the process control mechanism may be a valve which is used to adjust the fuel delivered to a furnace which heats the tubes of catalyst. Control of the fuel delivered to the furnace (e.g., by increasing or decreasing the fuel) may change the temperature of the furnace and the tubes of catalyst. Examples of process variables which may be measured, process variables which may be modified, and methods of modifying the process variables using process control mechanisms are described below in greater detail. The process 200 may continue during operation of the plant 100.

### The Controller

In one embodiment of the invention, the controller 140 may be a multi-variable controller. As a multi-variable controller the controller 140 may receive multiple process variables (e.g., hydrogen concentration, concentration of other gases, temperatures, pressures . . . ) as inputs and modify one or more process variables (e.g., process temperatures, flow rates/gas concentrations, and other variables) which are provided as outputs. In some cases, where multiple variables are measured, different sampling rates may be utilized for each variable being measured. The outputs may be received by process control systems (e.g., by systems in one of the process units such as the SMR unit 104 or the PSA unit 114) which modify the process variables to achieve a desired result (e.g., optimal production of hydrogen and carbon monoxide).

In one embodiment, the controller 140 may be a predictive controller 140. Where the controller 140 is both a multi-variable controller and a predictive controller, the controller 140 may be referred to as a multi-variable predictive controller (MVPC). A predictive controller may make discrete-time measurements for one or more process variables and predict future values for the process variables based on the received measurements. For example, as depicted in FIG. 3, the controller 140 may make measurements x(t) and x(t+Δt) of a methane concentration at a point within the hydrogen production process of the plant 100 at times t and t+Δt. A non-predictive controller may utilize linear interpolation 302 to determine a value of the concentration between the measurements. Where a predictive controller is utilized, a more accurate estimation 304 of the concentration may be made, for example, using a model for the production process being measured. Thus, as depicted, the predictive controller may obtain a more accurate estimation 304 of a process variable at a given time, and thus provide more accurate control for the process. The estimation 304 may also reflect other process variables measured by the controller 140.

In one embodiment of the invention, the controller 140 may be utilized to ensure that one or more process variables are maintained within a desired limit (e.g., above a threshold value, below a threshold value, or within a threshold range). Because the controller 140 may receive multiple process variables and utilize a predictive analysis to accurately determine a state of the production process, the controller 140 may be able to more accurately maintain the one or more process variables within the desired limit than if the controller merely received measurements for a single process variable or did not utilize a predictive analysis. In order to provide proper control of process variable, to request and read measurements of process variables, and to output control signals for controlling aspects of the production process as described herein, the controller 140 may execute a program including a plurality of instructions which, when executed, control the process as described herein. The program may be stored, for example, in a computer-readable medium such as a hard-disk drive, a read-only memory (ROM), a programmable read-only memory (PROM), a flash memory, a compact disk read-only memory (CD-ROM), or any other computer-readable medium.

### Controlling Aspects of the Process

In one embodiment of the invention, the hydrogen sensor 130 and the controller 140 may be used to improve control of individual units in the plant 100 such as the SMR unit 104, the membrane separation unit 122, the cold box 128, the PSA unit 114, and to improve the overall plant-wide control of the HyCO process by controlling multiple individual units together. Embodiments describing exemplary control procedures are described in greater detail below.

### Controlling the SMR Unit

As described above, the SMR unit 104 may receive steam and methane. The steam and methane may be heated and reacted in the presence of a metal-based catalyst to produce carbon monoxide and hydrogen. In some cases, all of the steam and methane may not be perfectly reacted, causing steam, methane, and carbon dioxide (known as methane or carbon dioxide slip) to be output by the SMR unit 104 in addition to the carbon monoxide and hydrogen. In some cases, there may be a desire to operate the SMR unit 104 in a stable manner (e.g., by maintaining stable temperatures within the SMR unit 104) to increase the life time of SMR catalyst tubes (described below) and reduce costly maintenance.

To reform the steam and the methane, the steam and methane may be passed through tubes of catalyst which are heated via a furnace within the SMR unit 104. In order to properly and efficiently reform the steam and the methane, it may be important to maintain the tubes of catalyst at a proper temperature. To maintain the proper temperature, the heat (e.g., the head duty) of the furnace may be modified, for example, by controlling the amount of fuel supplied to the furnace. The furnace may receive fuel from both the natural gas received by the plant 100 and as offgas from the PSA unit 114, both of which may be regulated to maintain the tubes of catalyst at the proper temperature. If the temperature of the tubes is to be lowered, the amount of fuel supplied to the furnace may be reduced, and if the temperature of the furnace is to be increased, the amount of fuel supplied to the furnace may be increased.

To reform the steam and methane, it is important to provide appropriate ratios of steam and methane to the SMR unit 104 to ensure that the reaction of the steam and methane is properly balanced such that all of the methane is reacted with all of the steam. The ratio of steam and methane within the SMR unit 104 may measured using a metric referred to as the carbon/hydrogen (C/H) ratio. A desired C/H ratio may be maintained by adjusting the steam and methane flow rates to regulate their ration and/or their total flow rate in the SMR unit 104. For example, in some cases, the methane may be provided from the amine unit 106 and/or as offgas from the PSA unit 114. To increase the C/H ratio, gas flow from the amine unit 106 and/or PSA unit 114 may be decreased. To decrease the C/H ratio, gas flow from the amine unit 106 and/or PSA unit 114 may be increased. The regulation of the furnace temperature and the C/H ratio within the SMR unit 104 may be referred to as load and temperature management (LTM).

In one embodiment of the invention, the temperature of the furnace and the C/H ratio may be adjusted by sampling the hydrogen concentration at an outlet of the SMR unit 104 and using the measured concentration to adjust the furnace temperature and/or the C/H ratio. The hydrogen concentration may be measured using a palladium-based hydrogen sensor 130 with a sampling rate which is sufficient to provide accurate control of the furnace temperature and/or the C/H ratio. The increased accuracy of control may have the additional benefit of creating more stable load and temperature management and allowing the SMR unit 104 to be operated more efficiently. Such increased accuracy of control may also result in fewer temperature variations on the tubes of catalyst within the SMR unit 104, thereby reducing breakage of the tubes due to changes in the tube temperatures.

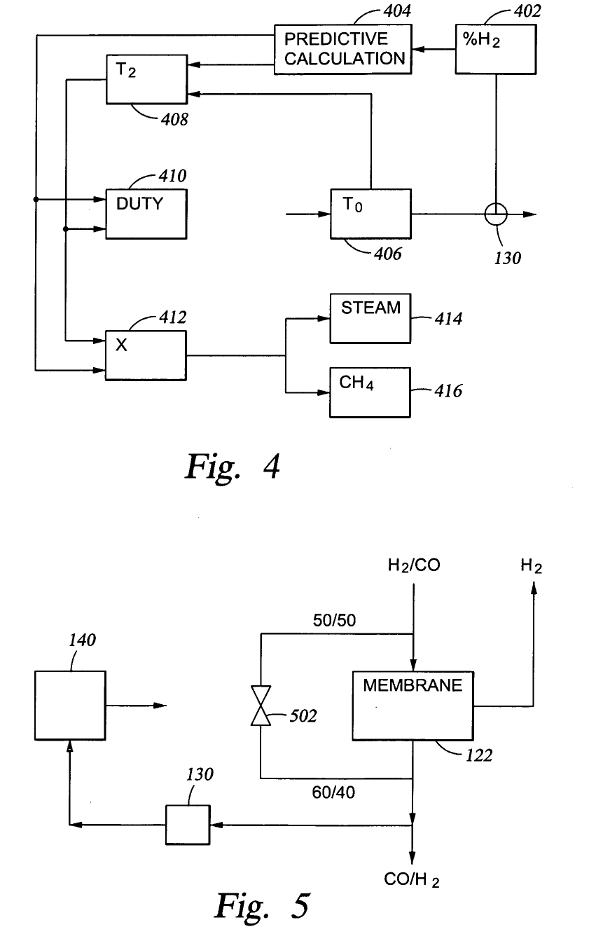

FIG. 4 is a block diagram depicting the control of process variables in the SMR unit 104 according to one embodiment of the invention. As depicted, the hydrogen sensor 130 may be used to measure a hydrogen concentration 402 at the output of the SMR unit 104. As described above, the sensor 130 may perform a direct measurement of the hydrogen concentration at the output of the SMR unit 104 (for example, without requiring an intermediate calculation to determine the hydrogen concentration in the gas mixture at the output). The measured hydrogen concentration, in addition to one or more other variables such as the threshold temperature T₀ 406 set by an operator of the SMR unit 104, the temperature of the tubes of catalyst T₂ 408, the current heat duty of the furnace 410, and the C/H ratio 412 to perform a predictive calculation and determine whether the process variables within the SMR unit 104 are within desired constraints. The calculation may be performed, as described above, using a multi-variable predictive controller 140. If the process variables are not within desired constraints, the controller 140 may modify one or more of the variables, for example, by changing the heat duty 410 of the SMR unit 104 (e.g., by changing the amount of fuel provided to the furnace) and/or changing the C/H ratio 412 by changing the steam flow 414 and/or the methane flow 416 into the SMR unit 104. The C/H ratio 412 may also be modified by controlling the carbon dioxide flowing back the SMR unit 104 from the amine scrubber 106. Other process variables within the SMR unit 104 may also be measured and modified as desired. As described above, by obtaining more frequent measurements of the hydrogen concentration 402 at the output of the SMR unit 104, more accurate control, and therefore greater efficiency, may be provided for the SMR unit 104.

### Control of Membrane Separation System

As described above, a membrane separation unit 122 is typically used in a HyCO plant 100 for achieving an accurate ratio of carbon monoxide to hydrogen (CO/H₂) in a gas stream. FIG. 5 is a block diagram depicting control of a membrane separation unit 122 according to one embodiment of the invention. As depicted, the outlet concentration of hydrogen and carbon monoxide may be controlled using a bypass valve 502. In one embodiment of the invention, the hydrogen concentration at the output of the membrane separation unit 122 may be measured using the hydrogen sensor 130.

The measured concentration may then be input into the controller 140 which may determine whether a desired concentration of hydrogen is present at the output of the membrane separation unit 122. For example, a ratio of hydrogen to carbon monoxide may be 50/50 at the input of the membrane separation unit 122 whereas a desired ratio of carbon monoxide to hydrogen at the output of the membrane separation unit 122 may be 60/40. If the hydrogen concentration detected by the sensor 130 is too large at the output of the membrane separation unit 122, then the bypass valve 502 may be closed by the controller 140 to reduce the concentration of hydrogen. If, however, the concentration of hydrogen is too small at the output of the membrane separation unit 122, then the bypass valve 502 may be opened by the controller 140 to increase the concentration of hydrogen. Because the hydrogen sensor 130 may provide decreased latency in obtaining hydrogen concentration measurements as described above, the bypass valve 502 may be more accurately controlled such that the desired ratio of carbon monoxide to hydrogen is maintained at the output of the membrane separation unit 122. Furthermore, when the process downstream from the membrane separation unit 122 includes a cold box 128, maintaining stable control of the carbon monoxide to hydrogen ratio may have the advantage of stabilizing the control of a hydrogen expander within the cold box 128.

### Controlling Other Units and Plant-Wide Control

In one embodiment, the hydrogen sensor 130 and controller 140 may also provide improved control for other aspects of the plant 100. For example, the sensor 130 and controller 140 may provide improved operation of the cold box 128 through better stability and operability of heat exchangers and expanders within the cold box 128 by measuring the hydrogen concentration of various streams in the cold box 128. Improved operation of the cold box 128 may provide energy savings and enhanced productivity.

The controller 140 and sensors may also provide improved operation of the PSA unit 114 through better separation control and improved throughput of the PSA unit 114. For example, the hydrogen sensor 130 may be used to monitor the level of hydrogen in the input of the PSA unit 114. The concentration of hydrogen measured using the hydrogen sensor 114 may then be used to adjust the time scale of each step of the PSA process for the best possible efficiency.

While described above with respect to control and optimization of multiple units such as an SMR unit 104, membrane separation unit 122, cold box 128, and PSA unit 114, embodiments of the invention may also provide for control of the plant 100 which integrates hydrogen concentration measurements from multiple hydrogen sensors 130 and multiple process variables using a controller 140 which monitors each of the measurements and variables and adjusts operation of the plant 100 for optimal production.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method for controlling production of hydrogen, the method comprising:
a) measuring an amount of hydrogen present at a first point in a hydrogen production process using a palladium-based hydrogen sensor;
b) measuring one or more first production process variables for the hydrogen production process using an additional sensor;
c) inputting the measurements of the amount of hydrogen and the one or more additional production process variables into a process controller; and
d) modifying one or more second production process variables for the hydrogen production process using a process control system.

2. The method of claim 1 wherein the one or more first production process variables include one of a temperature, a carbon/hydrogen ratio, and a carbon monoxide/hydrogen ratio.

3. The method of claim 1, the one or more second production process variables are the one or more first production process variables.

4. The method of claim 1, wherein the process control system includes one of a system for controlling a furnace heat in a steam methane reformer, a hydrocarbon feed flow and a steam flow in a steam methane reformer, and a flow rate in a bypass valve for a membrane separation unit.

5. The method of claim 1, wherein the palladium-based hydrogen sensor includes one of a palladium-nickel alloy and a palladium-gold alloy.

6. The method of claim 1, wherein the palladium-based hydrogen sensor includes a heating element.

7. The method of claim 1, further comprising:
e) calibrating the sensor by adjusting a temperature of the sensor to a temperature which is above a threshold temperature for correct measurement of hydrogen in a gas mixture including hydrogen and carbon monoxide.

8. The method of claim 1, wherein the palladium-based hydrogen sensor includes an additional layer which utilizes size-exclusion of molecules to prevent the sensor from measuring the presence of carbon monoxide and other impurities.

9. A computer-readable medium including a program which, when executed by a processor, performs a method for controlling production of hydrogen, the method comprising:
a) measuring an amount of hydrogen present at a first point in a hydrogen production process using a palladium-based hydrogen sensor;
b) measuring one or more first production process variables for the hydrogen production process using an additional sensor;
c) inputting the amount of hydrogen and the one or more additional production process variables into a process controller; and
d) modifying one or more second production process variables for the hydrogen production process using a process control system.

10. The computer-readable medium of claim 9 wherein the one or more first production process variables include one of a temperature, a carbon/hydrogen ratio, and a carbon monoxide/hydrogen ratio.

11. The computer-readable medium of claim 9, the one or more second production process variables are the one or more first production process variables.

12. The computer-readable medium of claim 9, wherein the process control system includes one of a system for controlling a furnace heat in a steam methane reformer, a methane flow and a steam flow in a steam methane reformer, and a flow rate in a bypass valve for a membrane separation unit.

13. The computer-readable medium of claim 9, wherein the palladium-based hydrogen sensor includes one of a palladium-nickel alloy and a palladium-gold alloy.

14. The computer-readable medium of claim 9, wherein the palladium-based hydrogen sensor includes a heating element.

15. The computer-readable medium of claim 9, wherein the method further comprises:
e) calibrating the sensor by adjusting a temperature of the sensor to a temperature which is above a threshold temperature for correct measurement of hydrogen in a gas mixture including hydrogen and carbon monoxide.

16. The computer-readable medium of claim 9, wherein the palladium-based hydrogen sensor includes an additional layer which utilizes size-exclusion of molecules to prevent the sensor from measuring the presence of carbon monoxide.

17. A system, comprising:
a) a palladium-based hydrogen sensor; and
b) a multi-variable predictive controller configured to:
i) measure an amount of hydrogen present at a first point in a hydrogen production process using a palladium-based hydrogen sensor;
ii) measure one or more first production process variables for the hydrogen production process using an additional sensor;
iii) input the amount of hydrogen and the one or more additional production process variables into a process controller; and
iv) modify one or more second production process variables for the hydrogen production process using a process control system.

18. The system of claim 17 wherein the one or more first production process variables include one of a temperature, a carbon/hydrogen ratio, and a carbon monoxide/hydrogen ratio.

19. The system of claim 17, the one or more second production process variables are the one or more first production process variables.

20. The system of claim 17, wherein the process control system includes one of a mechanism for controlling a furnace heat in a steam methane reformer, a methane flow and a steam flow in a steam methane reformer, and a flow rate in a bypass valve for a membrane separation unit.

21. The system of claim 17, wherein the palladium-based hydrogen sensor includes one of a palladium-nickel alloy and a palladium-gold alloy.

22. The system of claim 17, wherein the palladium-based hydrogen sensor includes a heating element.

23. The system of claim 17, wherein the multi-variable predictive controller is further configured to:
e) calibrate the sensor by adjusting a temperature of the sensor to a temperature which is above a threshold temperature for correct measurement of hydrogen in a gas mixture including hydrogen and carbon monoxide.

24. The system of claim 17, wherein the palladium-based hydrogen sensor includes an additional layer which utilizes size-exclusion of molecules to prevent the sensor from measuring the presence of carbon monoxide.
